(19) United States
(12) Patent Application Publication
(10) Pub. No.: US 2026/0276611 A1
(43) Pub. Date: Sep. 17, 2026

(54) SELF-POWERED NITROGEN DIOXIDE SENSING SYSTEM WITH EXCELLENT PERFORMANCE

(71) Applicant: China University of Petroleum (East China), Qingdao (CN)

(72) Inventors: Dongzhi Zhang, Qingdao (CN); Hao Zhang, Qingdao (CN); Yuncheng Ji, Qingdao (CN); Enxiu Wu, Qingdao (SD); Qingzhong Xue, Qingdao (CN); Wei Ke, Qingdao (CN); Liang Zhu, Qingdao (CN); Zhaofeng Wu, Qingdao (CN)

(73) Assignee: China University of Petroleum (East China), Qingdao (CN)

(21) Appl. No.: 19/566,969

(22) Filed: Mar. 13, 2026

(30) Foreign Application Priority Data
Mar. 17, 2025 (CN) 202510310931.X

(51) Int. Cl. G01N 33/00; G01N 27/12

(52) U.S. Cl. CPC G01N 33/0037; G01N 27/125

(57) ABSTRACT

A self-powered nitrogen dioxide sensing system with excellent performance is composed of an EMC-TENG and a MoS₂/MoO₃-based NO₂ gas sensor connected through a rectification and voltage regulation module, where the rectification and voltage regulation module consists of a bridge rectifier circuit, an RC filter circuit, and a 24 V Zener diode, the input terminal of the rectification and voltage regulation module is connected to the EMC-TENG to receive an alternating current signal, and the output terminal of the rectification and voltage regulation module is connected to the MoS₂/MoO₃-based NO₂ gas sensor to supply power to the gas sensor.

### CROSS REFERENCE TO RELATED APPLICATIONS

The present disclosure claims the priority to the Chinese patent application with the filing No. 202510310931.X, entitled “SELF-POWERED NITROGEN DIOXIDE SENSING SYSTEM WITH EXCELLENT PERFORMANCE” and filed on Mar. 17, 2025 with the Chinese Patent Office, the contents of which are incorporated herein by reference in their entirety.

### TECHNICAL FIELD

The present disclosure relates to the technical field of sensors, and more specifically to a self-powered nitrogen dioxide sensing system with an excellent performance.

### BACKGROUND ART

With atmospheric pollution becoming increasingly severe and global air quality continuing to decline, monitoring of atmospheric pollutants is urgently needed. Nitrogen dioxide (NO₂), one of the major atmospheric pollutants, primarily originates from high-temperature combustion processes such as industrial production, vehicle exhaust emissions, and fossil energy combustion. Excessive NO₂ not only causes photochemical smog, disrupts the ozone layer balance, and forms acid rain, resulting in severe environmental pollution, but also damages the respiratory system and lung, seriously endangering human health. According to data from the National Institute for Occupational Safety and Health (NIOSH) in the United States, NO₂ with the high concentration exceeding the exposure limit of 1 ppm poses a serious threat to life and health. Therefore, it is crucial to achieve efficient and stable monitoring of NO₂ for improving environmental quality and protecting human health.

Traditional NO₂ gas sensors require an external power source to maintain operation, and in order to achieve good sensing performance, high-temperature operating conditions are required to be created, increasing the operating cost and complexity of the sensors. Therefore, developing a gas sensor with excellent sensitivity at the room temperature and solving the power supply problem thereof have become the top priority for air quality monitoring. In 2012, Zhonglin Wang's team first developed a triboelectric nanogenerator (TENG) for mechanical energy harvesting, which converts, based on the coupling effect of electrostatic induction and triboelectric charging, energy generated by the human body, machinery, wind, and waves into electrical energy. With its diverse operating modes, low manufacturing cost, and flexible structural design, the TENG has become strong candidates for the power supply module for the self-powered gas sensor. In recent years, self-powered gas sensing systems have become an emerging research field, effectively solving the problems of sensors needing to rely on external power sources to maintain operation and battery replacement. However, how to improve the performance of self-powered gas sensing systems has become a new challenge for researchers. Currently, the performance of the self-powered gas sensor may be improved from two perspectives: on the one hand, by enhancing the output stability of the power supply module and on the other hand, by improving the sensitivity performance of the sensing module. For example, Zhang et al. used a template method to prepare a triboelectric nanogenerator with a blade structure. This method increased the generator's output voltage by 1.44 times and output current by 1.67 times, enabling stable driving of a nitrogen dioxide gas sensor. The MXene/WO₃ self-powered sensing system developed by Wang et al. utilizes MXene as a charge-conducting layer to accelerate electron transfer in the reaction, improve the gas adsorption and desorption rates, and significantly enhance the response performance of the NO₂ sensor at the room temperature.

Therefore, it is necessary to combine improving the output stability of the generator with improving the sensitivity of the sensor to enhance the sensing performance of the self-powered sensing system.

### SUMMARY

In view of this, the present disclosure provides a self-powered nitrogen dioxide sensing system with excellent performance. The present disclosure utilizes a triboelectric nanogenerator (EMC-TENG, Ecoflex™/MoS₂/MWCNTs-triboelectric nanogenerator) based on Ecoflex™/MoS₂/MWCNTs and PU skin cotton (PU cotton fabric) as the friction layer material (Ecoflex™ refers to thermoplastic elastomer materials with Ecological?Flexibility), the triboelectric nanogenerator exhibiting excellent output stability and anti-interference capabilities and having the output voltage and power density up to 1072V and 4.07 W/m², and prepare through a partial oxidation method a MoS₂/MoO₃ composite material as the gas-sensitive material for the room-temperature NO₂ sensor. The two are connected via a voltage conversion circuit, and a highly efficient and stable self-powered gas sensing system (EMCNS) is constructed based on impedance matching effects. This sensing system achieves excellent sensing performance (Vₐ/V_{g}=920% @100 ppm@17 s/12 s) under the room temperature condition without the aid of an external energy source. The gas sensing system with excellent sensing performance, good long-term stability, simple operation and no need of maintenance of the present disclosure paves the way for the construction of air quality monitoring sensor networks.

To achieve the above objectives, the present disclosure adopts the following technical solutions.

A self-powered nitrogen dioxide sensing system with excellent performance consists of an EMC-TENG and a MoS₂/MoO₃-based NO₂ gas sensor connected by a rectification and voltage regulation module,
where the rectification and voltage regulation module consists of a bridge rectifier circuit, an RC (resistor-capacitor) filter circuit, and a 24V Zener diode, the input terminal of the rectification and voltage regulation module is connected to the EMC-TENG to receive an alternating current signal, and the output terminal of the rectification and voltage regulation module is connected to the MoS₂/MoO₃-based NO₂ gas sensor to supply power to the gas sensor.

Preferably, the EMC-TENG is prepared by the following method:
(1) adding 2-3 g of MoS₂/MWCNTs powder to the Ecoflex™ silicone matrix in a ratio of 1:1, then adding Ecoflex™ silicone curing agent, performing strong magnetic stirring for 10-20 min, ultrasonically processing for 30 min, and mixing the MC powder and Ecoflex™ uniformly; and
(2) spin-coating the uniformly mixed Ecoflex™/MoS₂/MWCNTs silicone through a spin coating machine onto a thin copper foil-coated oak board as the negative friction layer of EMC-TENG, and similarly, attaching PU skin cotton to the thin copper foil-coated oak board as the positive friction layer of EMC-TENG.

Preferably, the MoS₂/MoO₃-based NO₂ gas sensor is prepared by the following method:
adding an appropriate amount of ethanol to 40-60 mg of the MoS₂/MoO₃ composite material and grinding thoroughly, then spin-coating the MoS₂/MoO₃ solution onto an interdigitated electrode to form a layer of uniform nanofilm, and drying to obtain the MoS₂/MoO₃ composite nanomaterial NO₂ gas sensor.

Preferably, the MoS₂/MWCNTs powder is prepared by the following method:
adding MWCNTs to 40-60 ml of 0.5 mol/L glucose solution and ultrasonically dispersing for 1 h to obtain a uniformly dispersed MWCNTs solution, then adding 0.2-0.3 g of thiourea and 0.3-0.5 g of sodium molybdate dihydrate, continuing ultrasonication for 30 min, then transferring the dispersed solution to a 60 ml autoclave, heating at 180° C. for 24 h, naturally cooling to a room temperature, performing centrifugation treatment on the solution by using deionized water and ethanol, drying at 60° C. overnight, and then thoroughly grinding in an agate mortar to obtain the product.

Preferably, the MoS₂/MoO₃ composite material is prepared by the following method:
adding 100-200 mg of MoS₂ powder to 30 ml of deionized water, magnetically stirring for 2 h to uniformly disperse the MoS₂ powder in the deionized water, then adding 10 ml of H₂O₂ solutions of different concentrations to the MoS₂ dispersed solution, ultrasonically processing for 30 min, then performing centrifugation treatment on the dispersed solution, washing with deionized water and ethanol, drying at 60° C. for 12 h, and annealing at 500° C. for 2 h under the condition of nitrogen purging to obtain the MoS₂/MoO₃ composite nanomaterial.

Preferably, the MoS₂ powder is prepared by the following method:
adding 1-1.5 g of sodium molybdate dihydrate and 2-3 g of thiourea to 40 ml of deionized water, stirring magnetically for 2 h, transferring the uniformly mixed solution to a 60 ml autoclave, heating at 200° C. for 24 h, naturally cooling the product to room temperature, washing multiple times with deionized water and ethanol to remove impurities, and finally, drying at 60° C. for 12 h to obtain black MOS₂ powder.

As can be seen from the above technical solutions, compared with the prior art, the present disclosure has the following beneficial effects.

The present disclosure uses the triboelectric nanogenerator with the Ecoflex™/MoS₂/CNTs and PU skin cotton as friction layer materials, as the power supply module of EMCNS. EMC-TENG has excellent output performance (voltage of 1072 V, current of 79 μA, and output power 4.07 W/m²).

The MoS₂/MoO₃ composite material is prepared via a partial oxidation method, and a NO₂ gas sensor is fabricated therefrom as the sensing module of the EMCNS. The two modules are connected via a voltage conversion and regulation circuit to form the EMCNS system. This system exhibits high stability. During one month, the EMCNS system was tested once every five days, with NO₂ gas repeatedly injected for three times in each test, and the voltage response of the EMCNS remained almost unchanged and the voltage could be recovered to the initial value after the reaction. Meanwhile, due to the formation of the MoS₂/MoO₃ heterojunction and the presence of 3D sheet-like stacking, the EMCNS possesses excellent sensing performance, and can achieve a high response value, a wide monitoring range, and fast response/recovery time (Vₐ/V_{g}=920% @100 ppm@17 s/12 s) at the room temperature. The EMCNS also exhibits good linearity (R²=0.95593) and excellent sensitivity (K=8.62992).

EMCNS shows great application potential in environmental monitoring and green energy harvesting, and provides some reference for self-powered sensing devices.

### BRIEF DESCRIPTION OF DRAWINGS

To more clearly illustrate the technical solutions in the embodiments of the present disclosure or the prior art, the drawings required to be used in the description of the embodiments or the prior art will be briefly introduced below. Obviously, the drawings described below are only embodiments of the present disclosure. For those ordinarily skilled in the art, other drawings may be obtained based on the provided drawings without paying creative effort.

FIG. 1A shows a schematic view of the EMC-TENG preparation process, and

FIG. 1B shows a schematic view of the EMCNS testing platform.

FIG. 2A-2B show SEM images of MoS₂-loaded MWCNTs; FIG. 2C-2D show SEM images of MoS₂ nanosheets; and FIG. 2E-2F show SEM images of the MoS₂/MoO₃ composite material generated by partial oxidation.

FIG. 3 shows the XRD patterns of MoS₂/MWCNTs, MoS₂/MoO₃, and MoS₂.

FIG. 4A-4D show the XPS spectra of MoS₂/MoO₃, where FIG. 4A shows the full scan spectrum; FIG. 4B shows the Mo 3d spectrum; FIG. 4C shows the O 1 s spectrum; and FIG. 4D shows the S2p spectrum.

FIG. 5A shows a schematic view of the working mechanism of EMC-TENG; FIG. 5B shows the potential distribution at 0.5 cm displayed through COMSOL simulation; FIG. 5C shows the potential distribution at 3.5 cm displayed through COMSOL simulation; FIG. 5D-5E show effects of material doping on the output performance of the EMC-TENG; FIG. 5F shows effect of EMC-TENG area (1-80 cm²) on the output performance; and FIG. 5G-5H show effects of humidity and temperature on the output performance of the EMC-TENG.

FIG. 6A shows voltage stability of the EMC-TENG over 1500 s; FIG. 6B shows current stability of the EMC-TENG over 1500 s; FIG. 6C shows change trends in open-circuit voltage (Voc) and short-circuit current (Isc) of the EMC-TENG with a load resistance varying from 0Ω to 1 G Ω; FIG. 6D shows power density curve of the EMC-TENG; FIG. 6E shows the steady-state voltage output of the EMC-TENG under the voltage regulation module; and FIG. 6F shows charging curves of the EMC-TENG for different capacitors over 100 s.

FIG. 7A shows the relationship between hydrogen peroxide concentration and sensing response curve; FIG. 7B shows the sensing performance of EMCNS for different NO₂ concentrations at the room temperature; FIG. 7C shows the relationship curve between the voltage response of the EMCNS and NO₂ concentration; FIG. 7D-7E show the response and recovery curves of the EMCNS at NO₂ concentrations of 5 ppm and 50 ppm; FIG. 7F-7G show the repeatability test of the EMCNS at NO₂ concentrations of 10 ppm and 20 ppm; FIG. 7H shows the long-term stability test of the EMCNS; and FIG. 7I shows the selectivity test of the EMCNS.

### DETAILED DESCRIPTION OF EMBODIMENTS

The technical solutions of the embodiments of the present disclosure will be clearly and completely described below with reference to the drawings in the embodiments of the present disclosure. Obviously, the described embodiments are only some embodiments of the present disclosure, and not all embodiments. Based on the embodiments of the present disclosure, all other embodiments obtained by those ordinarily skilled in the art without paying creative effort fall within the scope of protection of the present disclosure.

### Example 1

Material preparation: Ecoflex™ 00-50 was purchased from Smooth-On company, USA. Sodium molybdate dihydrate (NaMoO₄·2H₂O), thiourea (CH₄N₂S), and D-anhydrous glucose (C₆H₁₂O₆) were purchased from Shanghai Macklin Biochemical Co., Ltd. Hydrogen peroxide (H₂O₂), multi-walled carbon nanotubes (MWCNTs) and ethanol (CH₃CH₂OH) were purchased from Sinopharm Chemical Reagent CO., Ltd. PU skin cotton was purchased from Qingdao, Shandong. All chemicals were ready for use without further purification.

This example provided a self-powered nitrogen dioxide sensing system with excellent performance, the preparation method of which specifically included:
synthesis of MOS₂/MWCNTs (MC): adding MWCNTs to 40 ml of 0.5 mol/L glucose solution and ultrasonically dispersing for 1 h to obtain a uniformly dispersed MWCNTs solution, then adding 0.25 g of thiourea and 0.4 g of sodium molybdate dihydrate, continuing ultrasonication for 30 min, then transferring the dispersed solution to a 60 ml autoclave, heating at 180° C. for 24 h, then naturally cooling to room temperature, performing centrifugation treatment on the solution by using deionized water and ethanol, drying at 60° C. overnight, and then thoroughly grinding in an agate mortar to obtain MC powder;
synthesis of sheet-like MoS₂: adding 1.2 g of sodium molybdate dihydrate (NaMoO₄·2H₂O) and 2.4 g of thiourea (CH₄N₂S) to 40 ml of deionized water, magnetically stirring for 2 h, transferring the uniformly mixed solution to a 60 ml autoclave, heating at 200° C. for 24 h, naturally cooling the product to room temperature, then washing multiple times with deionized water and ethanol to remove impurities, and finally, drying at 60° C. for 12 h to obtain black MOS₂ powder; and
synthesis of MoS₂/MoO₃: adding 150 mg of the MoS₂ powder prepared in the above steps to 30 ml of deionized water, magnetically stirring for 2 h to uniformly disperse the MoS₂ powder in the deionized water, then adding 10 ml of H₂O₂ solutions of different concentrations (0.5%, 1%, 1.5%, 2%, 2.5%) to the MoS₂ dispersed solution, ultrasonically processing for 30 min, then performing centrifugation treatment on the dispersed solution and washing with deionized water and ethanol, drying at 60° C. for 12 h, and then annealing at 500° C. for 2 h under the condition of nitrogen purging to obtain the MoS₂/MoO₃ composite nanomaterial.

### Manufacturing of EMC-TENG and EMCNS

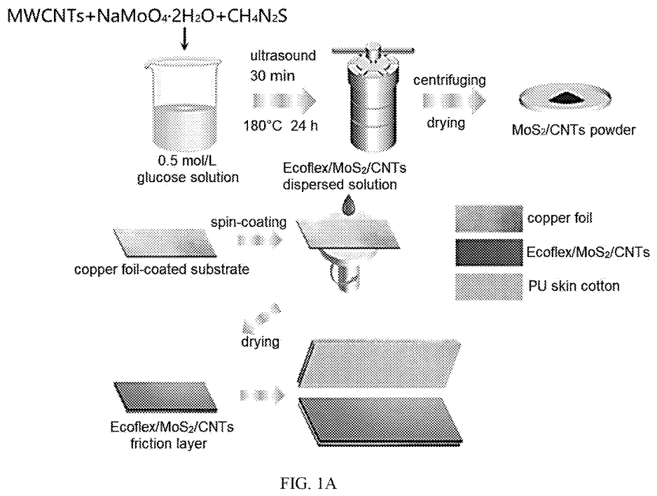

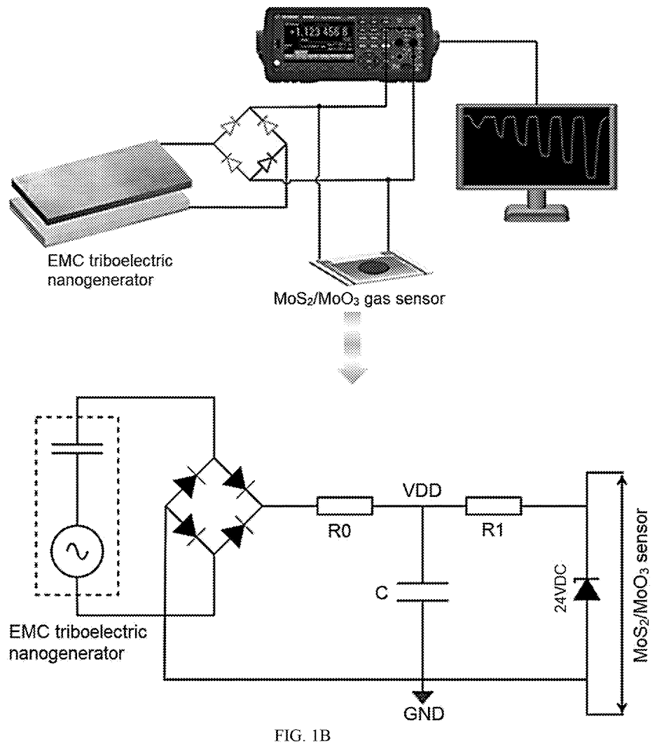

Manufacturing of EMC-TENG: adding 2.5 g of MC powder to the Ecoflex™ silicone matrix at a ratio of 1:1, adding Ecoflex™ silicone curing agent, performing strong magnetic stirring for 10 min, then ultrasonically processing for 30 min to uniformly mix the MC powder and Ecoflex™, spin-coating the uniformly mixed Ecoflex™/MoS₂/MWCNTs silicone through a spin coating machine onto a thin copper foil (electrode)-coated oak board (10 cm×5 cm) as the negative friction layer of EMC-TENG, and similarly, attaching PU skin cotton to the thin copper foil-coated oak board as the positive friction layer of EMC-TENG. The preparation process of EMC-TENG is as shown in FIG. 1A.

Fabrication of EMCNS: adding an appropriate amount of ethanol to 50 mg of MoS₂/MoO₃ composite material and thoroughly grinding, then spin-coating the MoS₂/MoO₃ solution onto an interdigitated electrode (5 mm×5 mm) to form a layer of uniform nanofilm, and drying to obtain the MoS₂/MoO₃ composite nanomaterial NO₂ gas sensor.

The EMCNS consisted of an EMC-TENG and a MoS₂/MoO₃-based NO₂ gas sensor connected by a rectification and voltage regulation module. The EMC-TENG with a size of 5×10 cm served as the power supply unit. The MoS₂/MoO₃-based NO₂ gas sensor with a size of 5×5 mm served as the sensing unit. The rectification and voltage regulation module was composed of a bridge rectifier circuit, an RC filter circuit, and a 24V Zener diode. The EMC-TENG converted the alternating current voltage to 24V direct current voltage through the rectification and voltage regulation module to supply power to the NO₂ gas sensor. The specific test platform is as shown in FIG. 1B.

### Measurement and Characterization Instruments

The output performance of the EMC-TENG and the gas-sensing performance of EMCNS for NO₂ were tested using a Keysight 34470A digital multimeter, and the surface morphology of the material was characterized using a scanning electron microscopy (SEM, NovaNanoSem 450). The crystal structure of the material was characterized using an X-ray diffraction instrument (XRD, RigakuMiniflex 600). The X-ray photoelectron spectroscopy (XPS, Thermo Scientific Escalab250Xi) was used to analyze the surface elemental and chemical composition of the samples.

### Sample Characterization

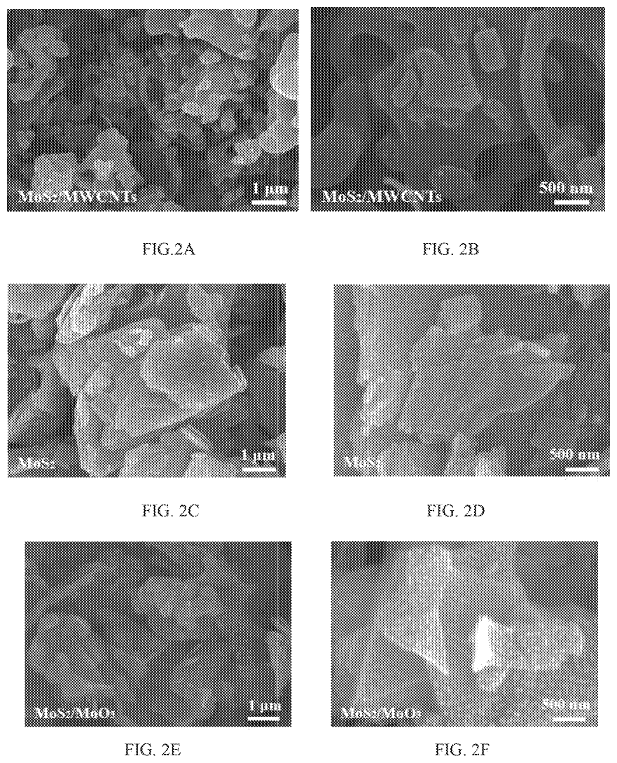

The surface morphology of the MoS₂/MWCNTs triboelectric material was observed using the scanning electron microscopy (SEM), as shown in FIGS. 2A and 2B. MoS₂ was successfully grown on the MWCNTs via self-assembly hydrothermal synthesis, confirming the successful preparation of MoS₂/MWCNTs. The surface morphology of the NO₂ gas-sensitive material was also observed using the scanning electron microscopy, as shown in FIG. 2C, indicating the successful preparation of sheet-like MoS₂. The sheet-like MoS₂ also exhibited a certain degree of stacking, effectively increasing the specific surface area of the gas-sensitive material and facilitating the adsorption of target gases on the surface of the gas-sensitive material. FIG. 2D further illustrates the sheet-like morphology feature of MoS₂. FIGS. 2E and 2F show the MoS₂ nanosheets oxidized with 1.5% H₂O₂ solution. It is worth noting that after H₂O₂ oxidation, dense nanoparticles were formed on the surfaces of the MoS₂ nanosheets, which were confirmed as MoO₃ nanoparticles by subsequent X-ray diffraction (XRD) analysis. This indicates that the H₂O₂ solution partially oxidizes the surface of MoS₂, resulting in formation of a surface heterostructure with MoO₃ nanoparticles. The presence of the heterostructure effectively enhances the interfacial effect and electron transport, promoting the adsorption and desorption processes of gases.

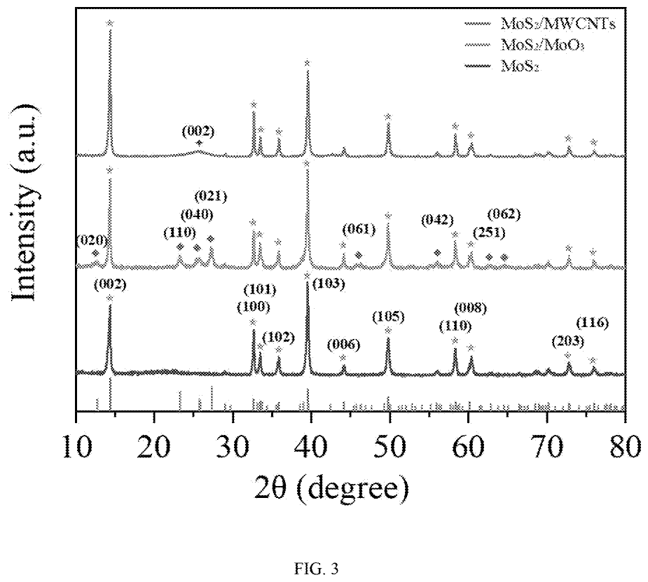

FIG. 3 shows the XRD patterns of the samples. The samples were scanned within a diffraction angle range of 10° to 80°. All diffraction peaks of the MoS₂ sample were consistent with the standard diffraction peaks of MoS₂ (JCPDS 37-1492). For the MoS₂/MoO₃ composite sample partially oxidized by H₂O₂, the diffraction peaks of MoS₂ overlapped with those of MoO₃, matching the standard diffraction peaks of MoO₃ (JCPDS 76-1003). Finally, the diffraction peaks of the MoS₂/MWCNTs sample were combination of the diffraction peaks of the MoS₂ and MWCNTs samples, conforming to the standard peaks (JCPDS 96-101-1061). XRD characterization further confirmed the successful preparation of the three samples above.

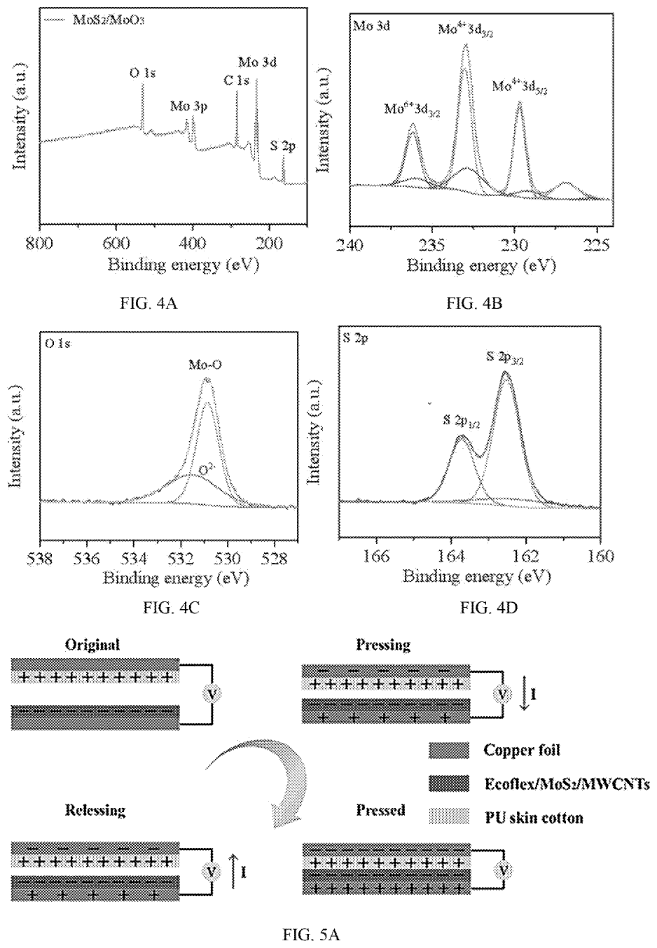

The chemical state and composition of partially oxidized MoS₂/MoO₃ composite material were investigated using X-ray photoelectron spectroscopy (XPS). FIG. 4A shows the full XPS spectrum of the MoS₂/MoO₃ heterostructure, confirming the presence of Mo, S, O, and C in the total spectrum. The C Is peak at 284.80 eV is typically used as a calibration reference peak. The Mo spectrum shown in FIG. 4B clearly distinguishes the peaks of Mo4+3d5/2, Mo4+3d3/2, and Mo6+3d3/2, and shows the S 2s peak associated with MoS₂ at position 226.8 eV. The presence of Mo6+ indicates that the sample contains some MoO₃. FIG. 4C shows that in the O 1 s spectrum, the peak at 530.9 eV corresponds to Mo—O, and the peak at 531.6 eV corresponds to O₂?. Their appearance may be attributed to lattice oxygen and surface adsorbed oxygen in MoO₃. FIG. 4D shows the peak distribution of S2p, with peaks at 162.5 eV and 163.6 eV corresponding to S2p_{3/2} and S2p_{1/2} respectively.

### Output Performance of EMC-TENG

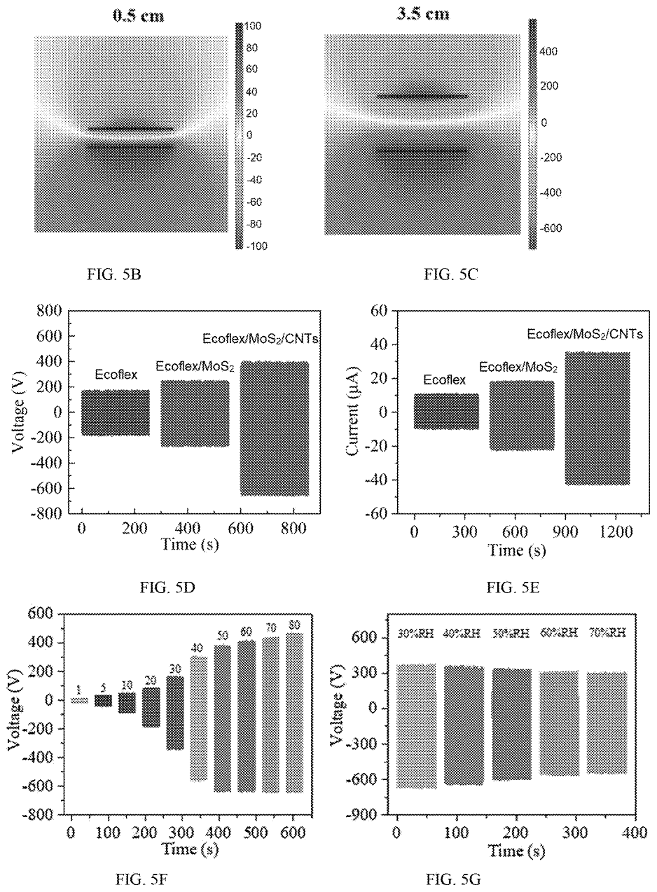

TENG have four working modes. EMC-TENG belongs to the vertical contact separation type, and its mechanism is as shown in FIG. 5A. According to the triboelectric material properties, the Ecoflex™/MoS₂/MWCNTs composite film has a stronger electron affinity than the PU film and is more likely to gain electrons, so it is a negative friction layer. Similarly, PU skin cotton is a positive friction layer.

When the positive friction layer of TENG comes into contact with the negative friction layer, due to the different electron affinities of the two friction layers, the induced charge may accumulate on the film surfaces of Ecoflex™/MoS₂/MWCNTs and PU skin cotton according to the polarity. (1) In the initial state, negative and positive charges are accumulated on the EMC composite film and the PU skin film respectively, the potential between the friction layers is balanced, and there is no potential difference between the electrodes.

(2) When the EMC and PU films are about to come into contact, a potential difference is generated between the two friction layers based on triboelectric charging, and at the same time, the presence of electrostatic induction causes electrons to flow from the bottom electrode to the top electrode, generating current.

(3) Once the EMC and PU films are in complete contact and electrostatic equilibrium is reached, there is no longer a potential difference between the electrodes, and no current flows into the external circuit.

(4) When the EMC and PU films are separated from each other, the external circuit generates a current in the direction opposite to that generated during contact. Therefore, when the EMC and PU are repeatedly in contact and separated, the EMC-TENG may generate an alternating current signal due to the repeated charge transfer.

In addition, the potential distribution of the EMC-TENG was studied through COMSOL, and the potential difference was directly proportional to the distance, as shown in FIGS. 5B and 5C. The effect of material doping on the output performance of the TENG was also investigated, where the output voltage and current of pure Ecoflex™ were 345.3 V and 21.3 μA, respectively, while after doping with MC composite powder, the output voltage and current of the EMC-TENG were significantly increased, up to 1072.4 V and 79 μA respectively, which are 3.11 times and 3.71 times that of the undoped composite material respectively, as shown in FIGS. 5D and 5E. This is because the doping of MC material increases the surface potential of the Ecoflex™ film, and the incorporation of MC effectively enhances the specific surface area of the composite material, generating charge traps, effectively enhancing the charge transfer rate and the generation of induced charges, and thus improving the output performance of the EMC-TENG. The effect of the friction layer area on the output performance was also studied, as shown in FIG. 5F.

The output voltages of the friction layers with areas from 1 to 80 square centimeters were discussed, to obtain that the output voltage increased at a relatively high rate for the areas from 1 to 50 square centimeters, while the output voltage increased at a relatively slow rate for the areas from 50 to 80 square centimeters. Therefore, 50 square centimeters (5 cm×10 cm) was selected as area of the positive and negative friction layers of the EMC-TENG, and the effects of temperature and humidity were studied, as shown in FIGS. 5G and 5H. It can be known form the figures that at 20° C. to 60° C., the output voltage of the EMC-TENG increased slightly with increasing temperature. At 30% RH to 70% RH, the output voltage of the EMC-TENG decreased in a slow trend. Owing to the moisture resistance of Ecoflex™, the EMC-TENG still maintained a high output voltage of 861.7V even at the humidity of 70% RH, and could provide stable power supply for the NO₂ sensor.

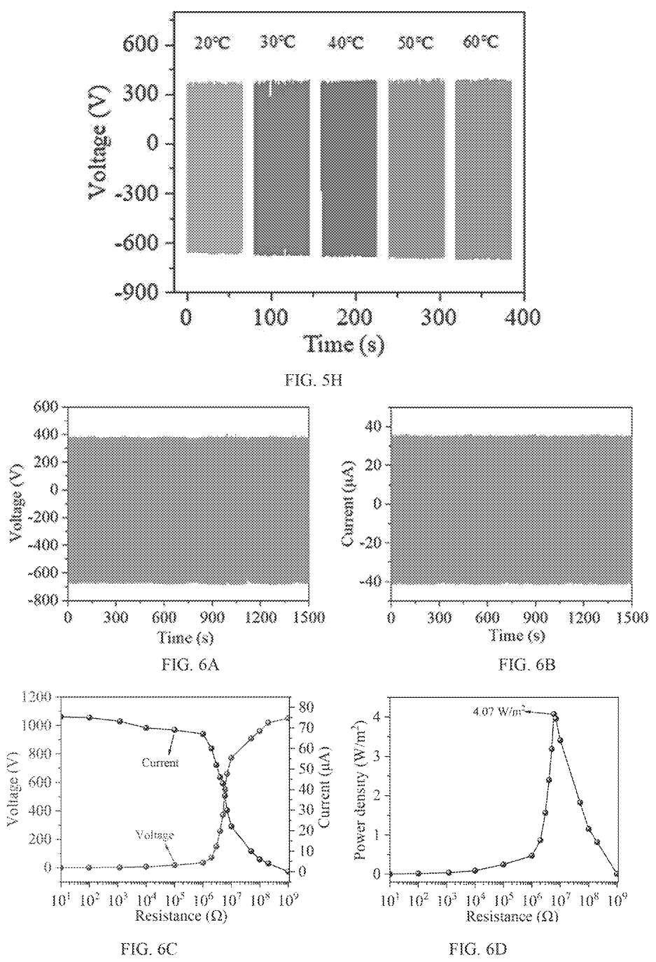

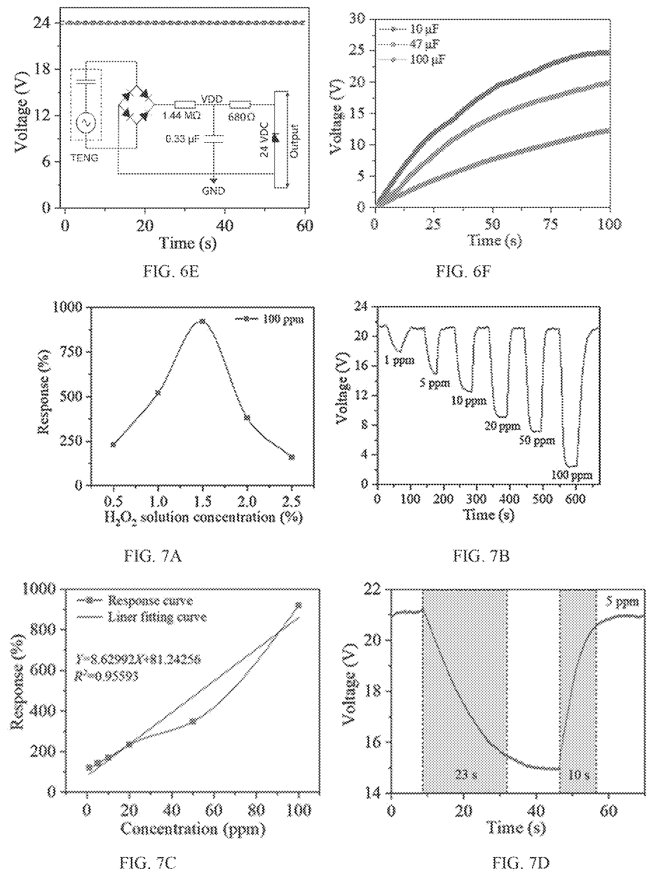

Besides anti-interference capability, stability is also one of the key indicators of whether the TENG can serve as a power supply module for a sensing system. To explore the stability of the EMC-TENG, the voltage stability and current stability over 1500 s and the output voltage stability over 30 days of the EMC-TENG were tested respectively, as shown in FIGS. 6A and 6B. As known from the data in the figures, the EMC-TENG possesses excellent mechanical stability. FIG. 6C shows the open-circuit voltage and short-circuit current curves of the EMC-TENG under load resistances varying from 0Ω to 1 G Ω. Due to Ohm's law, as the load resistance increases, the open-circuit voltage increases, and the short-circuit current decreases. Simultaneously, the power density P of the EMC-TENG under different load resistance was also calculated.

$$P = \frac{UI}{S}$$

where U represents the open-circuit voltage output of the EMC-TENG under a certain load resistance, I represents the short-circuit current output of the EMC-TENG under the corresponding load, and S represents the contact area between the two friction layers of the EMC-TENG. As shown in FIG. 6D, the figure shows the power densities of the EMC-TENG under different load resistances. When the load resistance is 6 MΩ, the power density reaches its maximum value of 4.07 W/m². the EMC-TENG can easily supply power to 50 LED lamps. As shown in FIG. 6E, 50 LED lamps are connected in series to form four letters TENG and powered and driven by the EMC-TENG.

To convert the alternating current signal to a direct current signal to supply power to the NO₂ gas sensor, a voltage conversion circuit was designed to stabilize the EMC-TENG's output voltage at 24V. The voltage conversion circuit and its conversion effect are as shown in FIG. 6F. Simultaneously, the charging capability of the voltage-regulated EMC-TENG for capacitors was tested. FIG. 6E shows the charging curves of the EMC-TENG at 10 μF, 47 μF, and 100 μF. It is worth noting that when the temperature and the humidity change, the regulated output voltage of the EMC-TENG remained at 24V, demonstrating extremely strong stability. This lays a solid foundation for its subsequent use as a power supply module for a self-powered NO₂ sensing system.

### Gas Sensing Performance of the EMCNS for NO₂

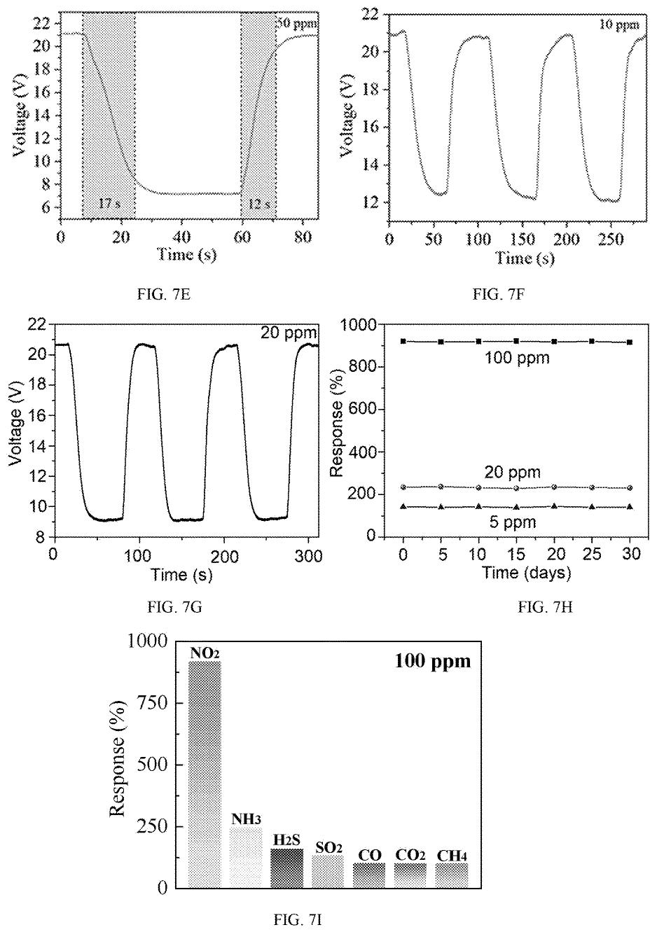

The EMCNS self-powered gas sensing system includes an EMC-TENG power supply module, a voltage regulation and conversion module, and a MoS₂/MoO₃-based NO₂ gas sensor. The sensing responses of the MoS₂/MoO₃-based NO₂ gas sensors prepared by partial oxidation with different concentrations of H₂O₂ were compared at a concentration of 100 ppm. As shown in FIG. 7A, when the H₂O₂ concentration is 1.5%, the maximum sensing response value of the EMCNS is 920%. This is attributed to more adsorption sites for NO₂ provided by the unique 3D sheet-like stacked structure and the formation of the MoS₂/MoO₃ heterostructure, which promotes gas adsorption and desorption. Simultaneously, the resistance variation range of the MoS₂/MoO₃ gas sensor was controlled within 1-10 MΩ, matching the load impedance range of the EMC-TENG at its maximum voltage change rate, thus conforming to the impedance matching effect and significantly improving the sensing performance of the EMCNS.

FIG. 7B shows the sensing performance of the EMCNS for NO₂ of concentrations of 1, 5, 10, 20, 50, and 100 ppm at room temperature. FIG. 7C illustrates the relationship between the EMCNS sensing response and the NO₂ gas concentration. It can be seen that the voltage response of the EMCNS is directly proportional to the NO₂ concentration. Furthermore, the voltage response curve of the EMCNS is subjected to linear fitting, and the result shows that the EMCNS has good linearity (R²=0.95593) and excellent sensitivity (K=8.62992). EMCNS has fast response/recovery time, which can meet the emergency applications.

As shown in FIGS. 7D and 7E, the response/recovery times of the EMCNS at NO₂ concentrations of 5 ppm and 50 ppm are respectively: 23 s/10 s and 17 s/12 s. Excellent stability is a prerequisite for system operation. This work investigated the operation of the EMCNS being repeatedly injected with gas and air for three times at NO₂ concentrations of 10 ppm and 20 ppm, as shown in FIGS. 7F and 7G. The EMCNS can perfectly replicate the first voltage response and recover the initial voltage value. As shown in FIG. 7H, the long-term stability of the EMCNS was also tested at concentrations of 5 ppm, 20 ppm, and 100 ppm, where the voltage response of the EMCNS was tested every 5 days, and within one month, the voltage response of the EMCNS showed almost no change. In summary, the EMCNS exhibits excellent performance in both short-term repeatability test and long-term stability test.

Finally, the response values of the EMCNS to different gases (NO₂, H₂S, SO₂, NH₃, CO, CO₂, CH₄) at a concentration of 100 ppm were tested. As shown in FIG. 7I, the response value of the EMCNS to NO₂ is much higher than those to the other gases, indicating the excellent selectivity of EMCNS to NO₂.

In this specification, the embodiments are described in a progressive manner, and each embodiment focuses on the differences from other embodiments. The same or similar parts between the embodiments may be referred to each other. For the device disclosed in the embodiment, since it corresponds to the method disclosed in the embodiment, the description is relatively simple, and the relevant parts may be referred to the method parts.

The above description of the disclosed embodiments enables one skilled in the art to implement or use the present application. Various modifications to these embodiments will be obvious to one skilled in the art, and the general principles defined herein may be implemented in other embodiments without departing from the spirit or scope of the present disclosure. Therefore, the present application will not be limited to the embodiments shown herein, but rather will conform to the widest scope consistent with the principles and novel features disclosed herein.

1. A self-powered nitrogen dioxide sensing system with excellent performance, composed of an EMC-TENG and a MoS₂/MoO₃-based NO₂ gas sensor connected through a rectification and voltage regulation module,
wherein the rectification and voltage regulation module is composed of a bridge rectifier circuit, an RC filter circuit, and a 24V Zener diode, an input terminal of the rectification and voltage regulation module is connected to the EMC-TENG to receive an alternating current signal, and an output terminal of the rectification and voltage regulation module is connected to the MoS₂/MoO₃-based NO₂ gas sensor to supply power to the gas sensor;
the EMC-TENG is prepared by the following method:
(1) adding 2-3 g of MoS₂/MWCNTs powder to a Ecoflex silicone matrix in a ratio of 1:1, then adding a Ecoflex silicone curing agent, performing strong magnetic stirring for 10-20 min, ultrasonically processing for 30 min, and mixing uniformly; and
(2) spin-coating a uniformly mixed Ecoflex/MoS₂/MWCNTs silicone through a spin coating machine onto a thin copper foil-coated oak board as a negative friction layer of the EMC-TENG, and similarly, attaching PU skin cotton to the thin copper foil-coated oak board as a positive friction layer of the EMC-TENG; and
the MoS₂/MoO₃-based NO₂ gas sensor is prepared by the following method:
adding an appropriate amount of ethanol to 40-60 mg of a MoS₂/MoO₃ composite material and grinding thoroughly, then spin-coating a MoS₂/MoO₃ solution onto an interdigitated electrode to form a layer of uniform nanofilm, and drying to obtain the MoS₂/MoO₃ composite nanomaterial NO₂ gas sensor.

2. The self-powered nitrogen dioxide sensing system with excellent performance according to claim 1, wherein the MoS₂/MWCNTs powder is prepared by the following method:
adding MWCNTs to 40-60 ml of 0.5 mol/L glucose solution and ultrasonically dispersing for 1 h to obtain a uniformly dispersed MWCNTs solution, then adding 0.2-0.3 g of thiourea and 0.3-0.5 g of sodium molybdate dihydrate, continuing ultrasonication for 30 min, then transferring a dispersed solution to a 60 ml autoclave, heating at 180° C. for 24 h, naturally cooling to a room temperature, performing centrifugation treatment on the solution by using deionized water and ethanol, drying at 60° C. overnight, and then thoroughly grinding in an agate mortar to obtain a product.

3. The self-powered nitrogen dioxide sensing system with excellent performance according to claim 2, wherein the MoS₂/MoO₃ composite material is prepared by the following method:
adding 100-200 mg of MoS₂ powder to 30 ml of deionized water, magnetically stirring for 2 h to uniformly disperse the MoS₂ powder in the deionized water, then adding 10 ml of H₂O₂ solutions of different concentrations to a MoS₂ dispersed solution, ultrasonically processing for 30 minutes, then performing centrifugation treatment on the dispersed solution, washing with deionized water and ethanol, drying at 60° C. for 12 h, and annealing at 500° C. for 2 h under a condition of nitrogen purging to obtain a MoS₂/MoO₃ composite nanomaterial.

4. The self-powered nitrogen dioxide sensing system with excellent performance according to claim 3, wherein the MoS₂ powder is prepared by the following method:
adding 1-1.5 g of sodium molybdate dihydrate and 2-3 g of thiourea to 40 ml of deionized water, stirring magnetically for 2 h, transferring a uniformly mixed solution to a 60 ml autoclave, heating at 200° C. for 24 h, naturally cooling a product to a room temperature, washing multiple times with deionized water and ethanol to remove impurities, and finally, drying at 60° C. for 12 h to obtain black MOS₂ powder.
